(19) United States
(12) Patent Application Publication
(10) Pub. No.: US 2017/0203072 A1
(43) Pub. Date: Jul. 20, 2017

(54) Detachable And Transportable CPAP Hose Holder

(71) Applicant: Kenneth Tonning, Manger (NO)

(72) Inventor: Kenneth Tonning, Manger (NO)

(73) Assignee: CrackNut AS, Isdalsto (NO)

(21) Appl. No.: 15/130,347

(22) Filed: Apr. 15, 2016

(30) Foreign Application Priority Data
Jan. 14, 2016 (AU) 2016200226

(51) Int. Cl. A61M 16/08; A61M 16/06; A61M 16/00

(52) U.S. Cl. CPC A61M 16/08; A61M 16/0057; A61M 16/06

(57) ABSTRACT

A CPAP hose holder device comprises a base, a hose entryway with one or more openings for a CPAP hose, entryway hose guides, an elongated support, support hose guides, and a platform. A bottom of the hose entryway is connected to the base and a top of the hose entryway is connected to a bottom of the support, and the platform is connected to a top of the support. The entryway hose guides are connected to the back of the hose entryway, and the support hose guides are attached to the back of the support. The hose guides and support are arranged to partially or fully enclose the CPAP hose and the support is substantially upright with respect to the base. The base and platform extend from the support in substantially the same direction.

### CROSS REFERENCE TO RELATED APPLICATION

The present application claims priority to Australian Patent Application Serial No. 2016200226, filed Jan. 14, 2016, which is hereby incorporated by reference herein.

### FIELD

The present disclosure relates to a CPAP hose holder device for the support of a CPAP hose and, more particularly, for the support a CPAP hose used with a CPAP machine. The present invention also relates to use of said CPAP hose holder, a method of moving of the CPAP hose to another position in the bed while sleeping, and a CPAP hose holder system. It further relates to relieving some of the inconvenience of sleep apnea treatment and associated methods, systems, and uses.

### BACKGROUND

Sleep apnea is a serious condition that causes individuals to repeatedly stop breathing as they sleep. The most common form is obstruction sleep apnea (OSA). When people afflicted with sleep apnea are asleep, the back of their throat muscles relax, and their airways close. The individuals will often wake up snorting, choking, or gasping for breath. Depending on the severity, this can occur hundreds of times in a night.

There are serious risks associated with sleep apnea. The constantly interrupted sleep reduces the amount of time the person spends in deep restful phases of sleep. Most people with sleep apnea are tired during the day time. Individuals afflicted with sleep apnea are shown to have a higher rate of high blood pressure, heart problems, diabetes, and liver problems. Additionally, sleep apnea can have a negative impact upon the quality of sleep for other people as well, with partners often sleeping in another room.

In cases where the sleep apnea is moderate to severe, the person will often be advised to use a continuous positive airway pressure (CPAP) machine. The individual wears a mask (nasal or nasal/oral), that is connected to a hose, which is connected to a CPAP machine. The machine will pump air through the hose, and to the individual, that is at a higher pressure than the surrounding air. This pressure prevents the upper airways from collapsing during sleep.

Although the CPAP system is a very effective and reliable treatment, many of those afflicted find it cumbersome and uncomfortable. When the individual is sleeping, the hose is usually placed on their chest or next to them in the bed. It is common for the hose to become stuck in the bedding or to the individual. When they roll over, or move in the bed, this can cause the hose to pull off the mask. This interrupts their sleep as the individual must reattach their mask, thereby defeating the purpose of using the CPAP system in the first place. This problem can be very frustrating and cause the person to stop using the device, thus risking their health. There have been a number of attempts to solve this problem of the hose dislodging the mask as the individual sleeps.

Some of these solutions require the attachment of permanent devices or structures to the headboard, wall, or ceiling. These solutions are not portable when the individual needs to travel. Additionally these structures can not be dissembled quickly if needed. They can also present a safety hazard if someone makes contact with them at speed. An individual may be unable or unwilling to attach things to their wall, ceiling, or headboard.

An individual can a place a stand on the floor. These devices tend to be large, unwieldly, and are difficult to move. Also, the holder's clinical appearance can create the additional problem of making the individual feel like they are in a hospital. The sheer size of these devices can make them difficult to travel with.

An individual can use devices that are wedged between the mattress and box springs. These take time to disassemble, particularly if the bed is up against a wall. It is not convenient to move these devices every time someone wishes to make or play in the bed. These solutions also do not function well when an individual goes camping and no box springs are available.

There are products on the market that route the CPAP machine hose into a special pillow. The individual then attaches their mask to the pillow by means of a second hose. This requires the user to decide before they sleep which side of the pillow they will sleep upon. When the individual rolls from one side of their body to the other, the mask can either be pulled off or the hose can lie uncomfortably across the user's face. Also, the individual has no choice but to take the special pillow with them when traveling.

A further problem with these solutions is how the hose is attached to the holder itself. These are often simply draped over the body of the holder. When the hose is attached, it is usually with only few clips between the holder and the hose. This makes the hose and holder system vulnerable when someone bumps it. This kind of treatment can be expected in many situations; for example: playing with children.

A major problem that most of these solutions have in common is that they are fixed in one position with respect to the bed. These solutions attempt to solve the problem of the individual turning from one side to the other side. But they fail to address if the individual wishes to move to a different location in the bed.

Individuals with larger beds and/or those with a partner in the same bed will move laterally on the bed as well. As the individual moves away from the point where the holder and hose connect, the chance increases of the mask dislodging. Previous solutions require that the individual decides where they are going to sleep in the bed before they go to sleep. If the individual moves to a different position in the bed, the tension between the hose holder and the mask can pull the mask off.

Therefore there is a need for a device to be used with a CPAP system that would reduce the number of times the mask is pulled off or the hose gets in the way, thereby increasing the individual's quality of sleep.

### SUMMARY

In one aspect, there is provided a CPAP hose holder device including a base, a hose entryway with one or more openings for a CPAP hose, entryway hose guides, an elongated support, support hose guides, and a platform, wherein: a bottom of the hose entryway is connected to the base and a top of the hose entryway is connected to a bottom of the support, and the platform is connected to a top of the support. The entryway hose guides are connected to the back of the hose entryway, and the support hose guides are attached to the back of the support. The hose guides and support are arranged to partially or fully enclose the CPAP hose. The support is substantially upright with respect to the base, and the base and platform extend from the support in substantially the same direction.

In accordance with one embodiment, the support is comprised of two or more separate parts, for example two. The support can be curved or straight, and is for example straight. The device may have a cover. The device is arranged to be partially or wholly dissembled. A pillow can be temporarily attached to the device

In another aspect, use of the CPAP hose holder device involves the prevention of face mask removal during sleep, wherein a hose from a CPAP machine is placed inside the device and a pillow is placed on the base of the device.

In another aspect, there is provided a method of moving the CPAP hose device including the following steps: (a) the individual lifts their head from their pillow; (b) the individual moves the CPAP hose holder and pillow together to a new position in the bed; (c) the individual places their head back upon the pillow.

In another aspect, there is provided a CPAP hose holder system comprising: (a) a CPAP hose holder device; (b) a pillow; (c) a CPAP machine; (d) a CPCP hose; and (e) a CPAP facemask.

Example embodiments of the CPAP hose holder provide a device used for the support of a CPAP hose during the treatment of sleep apnea. Example embodiments meet one or more of the deficiencies of previous solutions to the technical problems discussed above as well as other previously unmet needs. Example embodiments reduce the chance that the face mask will be removed because of movement while the individual sleeps; both by rolling over or by moving to a different location in bed.

Example embodiments are not affixed to a wall, table, headboard, ceiling, etc. This gives the individual flexibility in where they will use the device. It also reduces the hazards of impact with this device while in the assembled position.

Example embodiments are not very large. Example embodiments can be disassembled into several pieces and stored in a CPAP system travel bag. Additionally, the device can be broken down quickly. These factors, among others, make the device well suited for travel. These also make the device well suited for fast disassembly before making the bed or engaging in other activities where the presence of a CPAP hose or hose holder are undesirable.

The hose sits in an example embodiment of the present invention such that it provides both protection to the hose and helps the hose stay in place. The hose is partially hidden from view which gives improves upon the aesthetics, particularly when compared to previous solutions. In addition, the holder can be decorated to fit in with the surroundings, such as cartoon prints for a child.

Example embodiments can be used with any pillow. This allows the individual more flexibility as they travel. There is no need to take along special pillows. The device can also be used while camping.

Example embodiments allow the individual to decide where they are going to sleep in the bed. It is a simple matter to move the device during sleep by moving the pillow. This allows the individual to move their sleep location at will. It does not require them to decide where they are going to sleep in the bed when they go to sleep.

### BRIEF DESCRIPTION OF THE DRAWINGS

FIG. 1A discloses an isometric view from the front of an example embodiment of the disclosure.

FIG. 1B discloses an isometric view from the front of an alternate example embodiment of the disclosure.

FIG. 2 discloses an isometric view from the rear of the example embodiment of FIG. 1A.

FIG. 3 discloses a side view of the connection between the upper and lower supports.

FIG. 4 discloses a top view of the base.

FIG. 5 discloses a detailed front view of the lower support.

FIG. 6A discloses a detailed side view of the lower support.

FIG. 6B discloses a detailed side view of an alternate example embodiment of the disclosure.

FIG. 7 discloses a detailed side view of the upper support.

FIG. 8A discloses a detailed rear view of the upper support.

FIG. 8B discloses a detailed side view of an alternate example embodiment of the upper support.

FIG. 8C discloses a detailed side view of an alternate example embodiment of the upper support.

FIG. 9A discloses a detailed top view of the platform.

FIG. 9B discloses a detailed top view of an alternate example embodiment of the platform.

### DETAILED DESCRIPTION OF THE DRAWINGS

Using the attached drawings, the technical contents, and detailed descriptions, example embodiments of the present disclosure are described. Alternate embodiments will also be presented.

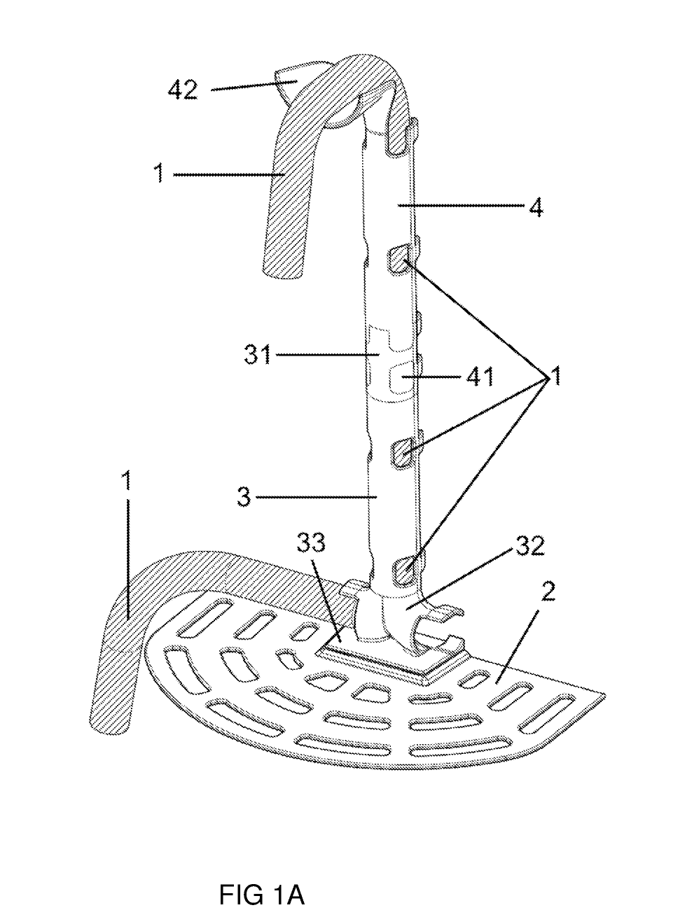

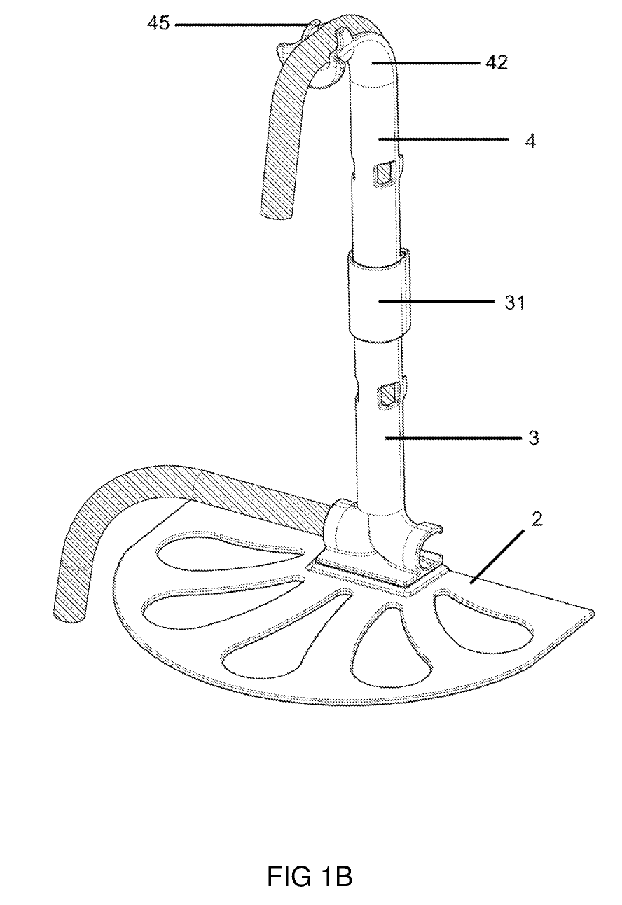

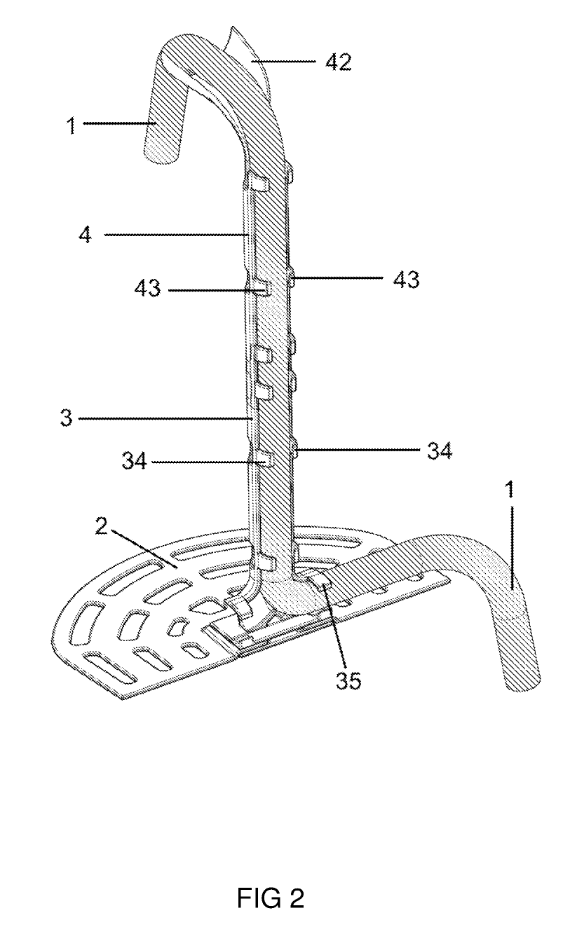

FIGS. 1A, 1B, and 2 disclose isometric views of an example embodiment of the invention. The hose 1 enters the example device through the hose entryway 32. The hose 1 is found inside the lower support 3. This lower support 3 is connected to the base 2 by a base connection means 33. The lower support 3 is connected to the upper support 4 by the lower connection means 31 and the upper connection means 41. The hose 1 rests upon the platform 42. The hose 1 is removably held inside an example embodiment of the disclosure by means of an entryway hose guides 35, lower hose guides 34, and upper hose guides 43. A pillow (not shown) is placed on top of the base 2. A CPAP face mask and CPAP machine (not shown) connects to the ends of the hose. The upper 4 and the lower 3 support combine to make a single support. In the embodiments shown, the hose entryway 32 has two openings where the hose can enter. But it is also possible to have a hose entryway 32 with one opening or with three or more openings.

FIG. 1B shows an alternate embodiment of the present invention. The hose rests upon a platform 42 that has hose guides 45. Additionally, it has a cross section profile similar to that of the top support 4. The upper connection means 41 fits entirely inside of the lower connection means 31 and is thus hidden from view. The base 2 show in FIG. 2 has a different number and size of holes than the base shown in other figures.

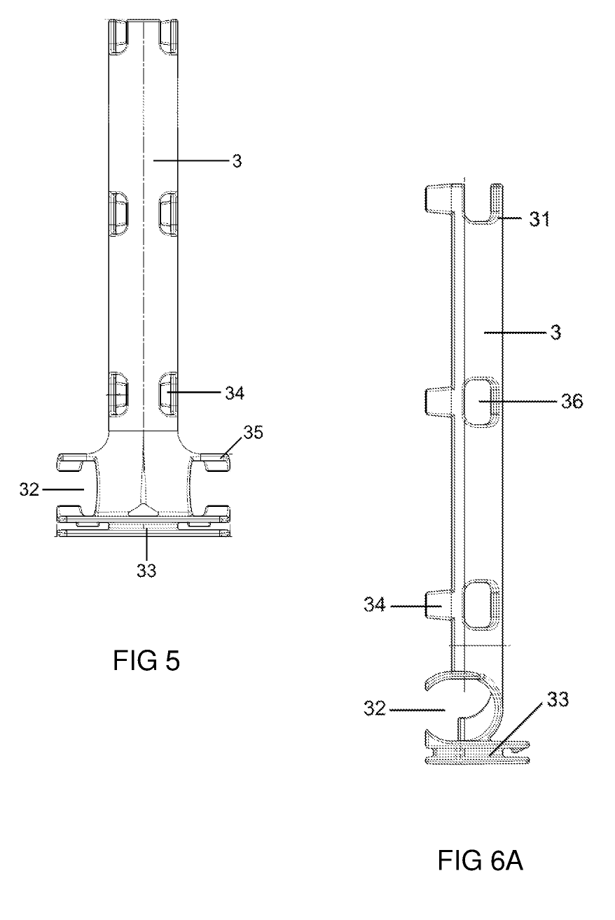

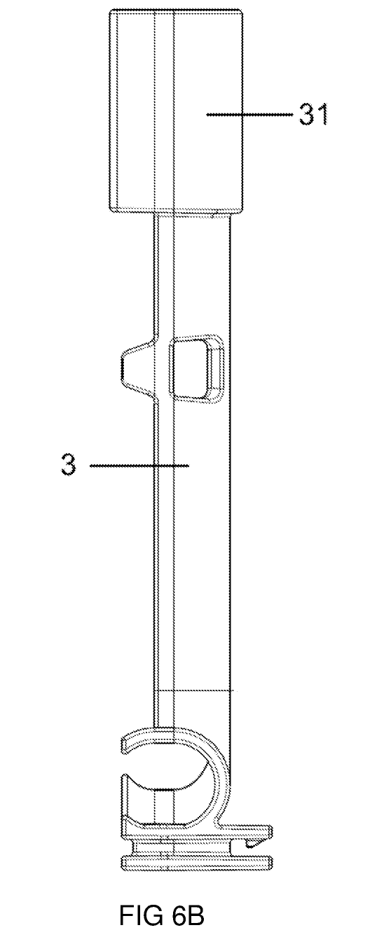

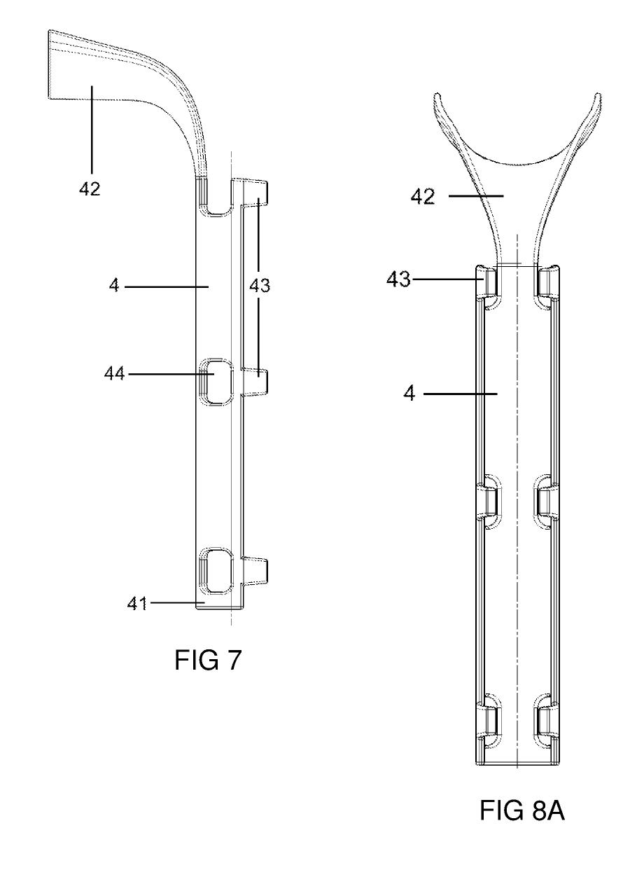

While the upper 4 and lower 3 supports are shown with holes 36 (FIG. 6A) and 44 (FIG. 7), an alternate embodiment could be without holes. FIGS. 1 and 2 show the lower connection means 31 on top of the upper connection means 41, but this is not the only possible configuration. The upper connection means 41 could lie on the lower connection means 31. Additionally, the connection between the upper support 4 and the lower support 3 could be achieved in a number of different ways. Examples include, but are not limited to, a threaded connection, clamps, Velcro, spring type quick connections, etc. The hose guides, 35 and 34 and 43, can be shaped different than the arcs that are depicted in the figures. Additionally they are not required to be immobile with respect to the lower support 3. Alternate embodiments of the hose guides of the present invention could include, but are not limited to: flexible, spring type, buckle, or latches. The shape of the platform 42 shown in the figures of example embodiments of the present disclosure can be different than that shown. Examples of different shapes include, but are not limited to, square, rectangle, circular, flat, curved, etc. While the back part of an example embodiment of the present invention is shown as open, an alternate embodiment could include a cover.

Hose guides hold and guide the CPAP hose. Herein “entry hose guides” refer to hose guides affixed to the entryway, while “support hose guides” refer to hose guides connected to the support. The hose guides 34, 35, 43 hold and guide the CPAP hose. The support 3, 4 forms the structure that the hose and guides are connected to and supported by. Usually the hose guides 34, 35, 43 only partially enclose the hose 1. Under normal circumstances, this is one embodiment. However, in some circumstances these advantages may be outweighed by other needs. For instance, if it is desired for aesthetic reasons for the hose to be as concealed as possible, the hose guides 34, 35, 43 can be constructed to fully encompass the hose 1. This may also be done by molding the hose guides and support as one piece, or by adding a protective sleeve to the outside of the device. Such a sleeve or fully hose-enclosing structure can in accordance with one embodiment be produced in an insulating material. This insulating material can for example be neoprene, Styrofoam or knitted fabrics, and would provide an advantage if the CPAP machine is to be used in low temperatures. During low temperatures, such as during the winter, it is a known problem that condensation forms in the hose. By providing insulation, this problem could be alleviated.

Example embodiments of the present disclosure can be made out of a large range of different materials. Examples include, but are not limited to: wood, metal, paper, plastic, or combinations thereof. In hospital use, it could be desirable for an example embodiment of the current invention to be made out of materials that can be sterilized, such as metal. Additionally, each piece of example embodiments of the present disclosure could be made out of the same or different material.

The figures show the support as two different pieces: the lower support 3 and the upper support 4. An alternative embodiment can be a support made of a single piece, or of more than two pieces.

Although the figures show the support as being made up of two support pieces (a lower and upper support) this is one example embodiment, but it is not the only embodiment of the present disclosure. This embodiment avoids having one long support piece when disassembling and travelling with the device, and makes it easier to pack. Other embodiments are to construct the support as one long piece, thereby making both the construction and assembly easier. The support could also be made of three or more pieces; allowing for more customization of the total support length. While a straight support is the embodiment shown, it can also be curved if needed. This would be useful in situations where the individual has very little space above their pillow.

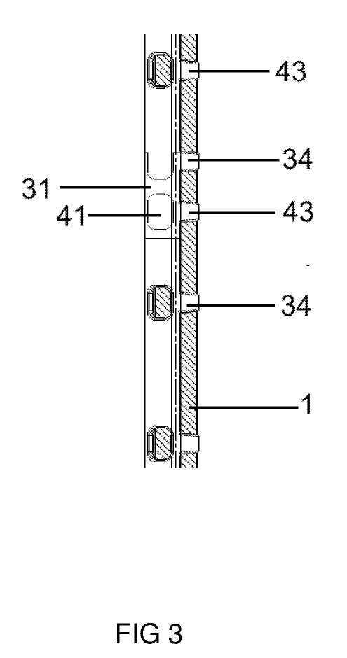

FIG. 3 discloses a side view of the connection between the upper 4 and lower 3 supports of an example embodiment of the present invention. The hose 1 is held in place by the lower hose guides 34 and the upper hose guides 43. The upper 4 and lower 3 supports are connected by means of the upper connection means 41 and the lower connection means 31.

The figures show hose guides, 34 and 43, as single molded pieces on the upper 4 and lower 3 supports. However, alternative embodiments could include hose guides that are more than a single piece. An alternate embodiment of the present disclosure could use straps, springs, clamps, or other mechanical means in the place of, or in addition to, the shown hose guides. In an alternate embodiment, the hose guides could be removable from the support.

The connection between the upper 4 and lower 3 supports of an example embodiment of the present disclosure is shown in FIG. 3 as the upper support 4 connection means sliding into the lower support 3. However, there are other alternatives. The lower support 3 could slide into the upper support 4. Other possible solutions include, but are not limited to, screwing any support pieces together, a clamping mechanism between the support pieces, a support telescoping from another support, and a hinge between the support pieces. The support pieces can be connected by one or more different connection means.

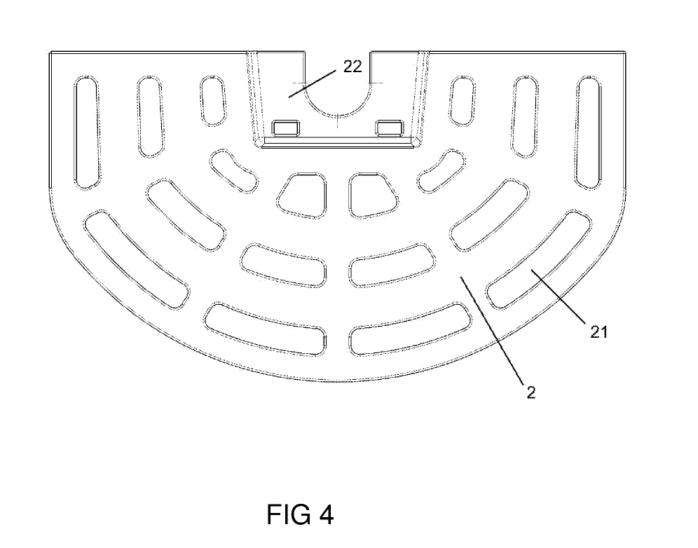

FIG. 4 discloses a top view of the base 2. The base 2 contains base holes 21. The base 2 is connected to the lower support 3 at the base connection section 22.

A pillow (not shown) can be placed upon the base 2. FIG. 4 shows a base plate with holes 21. Alternative embodiments could include a base with no holes. The holes make the base lighter, and allow for circulation. Additionally, the base could have texturing, improving its grip to the bed and/or pillow and reducing undesired sliding. The pillow could be attached, temporarily or permanently, to an example embodiment of the present disclosure by an attachment means or by base texturing. The attachment means could be connected to an example embodiment of the present invention at the base or other parts of the device. Additionally, a lip or protrusion could be placed at the back of an example embodiment of the present disclosure so it is partially held in place by the edge of the mattress.

In an example embodiment of the present disclosure, the base connection means 33 is slid onto the base connection section 22. There are several alternatives including, but not limited to, hinges, pins, threaded connections, and/or clamps. It is also possible that the connection between the connection means 33 and the connection section 22 be adjustable. This could be a type of connection that allows the hose entryway 32 to lean backward and forward and/or side to side with respect to the plane of the base 2.

Similar types of adjustment mechanisms could also be implemented at the connections between other parts of example embodiments of the present disclosure. These kinds of connections could be useful if a high degree of positional adjustment was desired.

Example embodiments of the present disclosure can be used with any pillow. There are cases, however, where the base size will need to be changed. An example of this would be a small pillow made for a child. The base is shown in FIG. 4 as a rounded rectangle, but a circular, square, rectangular, or other base shapes would be acceptable. The size and shape of the pillow and the size and shape of the base 2 should therefore be somewhat adjusted to each other. In one example, the base should not be so large as to protrude beyond the pillow, as this might be uncomfortable, unsightly, and would expose the base to unintended movement. However, the base also should be large enough to form a solid base in order for the support to stay upright during use. When in use, both the pillow and the head of the individual resting on the pillow aids in this, as their weight and friction will keep the base oriented correctly, i.e. flat on the bed so that the supports 3 and 4 are kept vertical. The base 2 should therefore be large enough for a head resting on the pillow to hold it in place.

While FIG. 4 shows that the base connection section 22 is centered on the rear part of the base 2, this choice of location is not the only choice. There are foreseeable situations where this connection will need to be made at a different place, for example: to work around the position of other equipment in the bed. The base connection section 22 could be found anywhere on the base plate, but preferably toward the rear of the base 2, most preferably centered toward the rear of the base.

FIGS. 5 and 6 disclose detailed views of the lower support 3. The entryway 32 is connected to an entryway hose guide 35. A base connection means 33 is connected to the bottom of the lower support 3. Lower hose guides 34 are also shown. There are holes 36 in the lower support 3. The top of the lower support 3 contains the lower connection means 31.

FIG. 6B discloses a detailed view of an alternative embodiment of the present disclosure with a different lower connection means 31 than that shown in FIG. 6A.

FIGS. 5, 6A, and 6B show a relatively small distance between the two different hose entryways 32; alternative embodiments could have distances that are larger or smaller than shown.

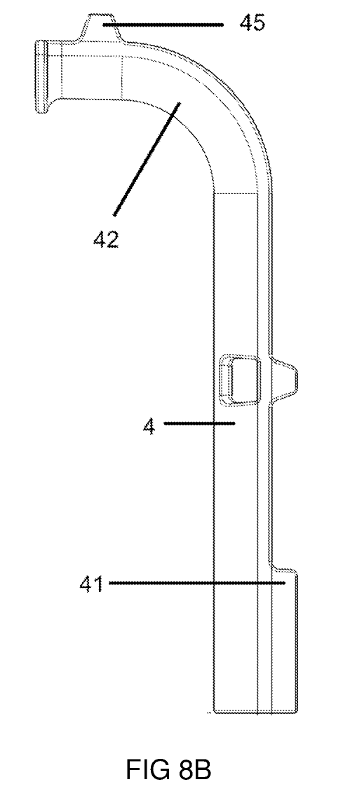

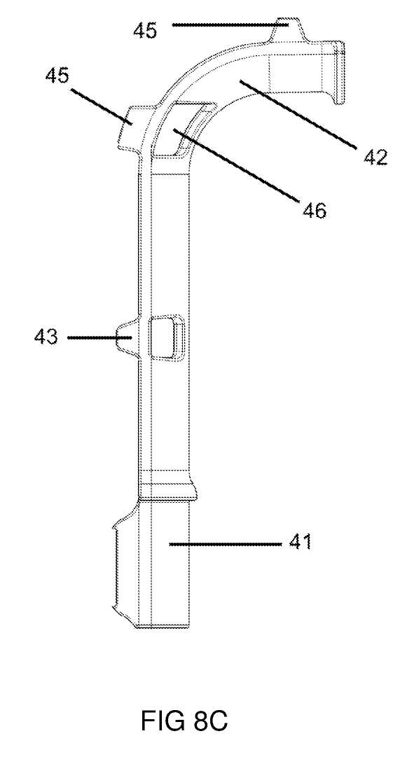

FIGS. 7 and 8A disclose detailed views of the upper support 4. The upper connection means 41 is found on the bottom part of the upper support 4. Holes 44 are found in the upper support 4. The upper hose guides 43 are connected to the upper support 4. The platform 42 is connected to the top part of the upper support 4.

FIG. 8B discloses a side view of an alternative embodiment of the upper support 4. It uses a different type of upper connection means 41. It also shows another possible shape for the platform 42 and the addition of a platform hose guide 45. FIG. 8C also discloses a side view of an alternative embodiment of the disclosure. In this embodiment, the platform 42 has a second platform hose guide 45. Also, the platform 42 has holes 46 and another embodiment of the upper connection means 41.

While the figures show the platform 42 as a flat surface, an alternative would be a platform with a clamp or slot for the CPAP hose 1.

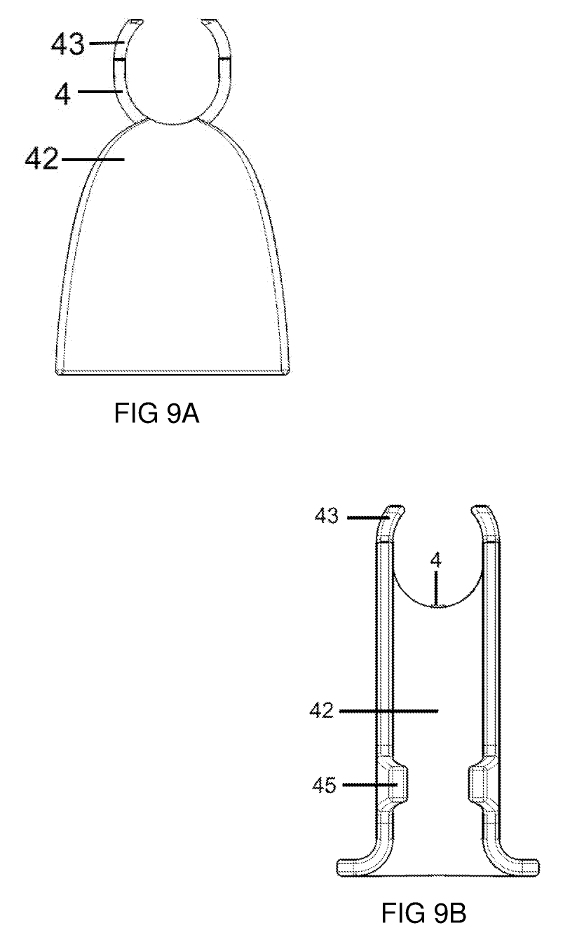

FIG. 9A discloses a detailed top view of the platform 42. The platform 42 is connected to the upper support 4. The upper hose guide 43 is connected to the upper support 4.

FIG. 9B discloses a top view of an alternative embodiment of the disclosure. In this embodiment, the platform 42 has the same width as the upper support 4. The platform hose guides 45 are connected to the platform 42.

While the figures show the platform 42 and the upper support 4 as a single molded piece, these could be two separate pieces.

Use of the CPAP hose holder for the prevention of face mask removal during sleep shall now be explained. The CPAP machine is connected to the face mask by a hose. The individual places the base 2 of an example embodiment of the present disclosure directly under the individual's pillow. Preferably the base 2 is placed under the pillow so that the support 3 protrudes upward from a position above the individual's head, although it could also be protruding from the left or right side of the individual's head. This hose 1 is connected to the CPAP machine and placed in an example embodiment of the present disclosure. The hose 1 exits the upper platform 42 and hangs freely down towards the individual's face. At the end of the hose 1, the facemask is connected. The individual sleeps with the facemask on, as normal. When the individual turns his head or otherwise moves his face, the hose, due to being free hanging above the individual, will not become tangled up, or be in the way or pulled upon, thus preventing face mask removal.

The method for the moving of the hose holder, while the CPAP hose is attached, during sleep is given by the following steps:
a) The individual lifts their head.
b) The individual moves the hose holder and pillow together to the desired location.
c) The individual places their head back upon their pillow.

Since the base 2 is flat and fitted under the pillow, it is easy to move with the pillow even if it is not attached thereto. Sliding the pillow around in bed while mostly asleep is therefore possible, greatly improving the ability of CPAP users to change position in bed and therefore sleep more naturally.

Even though the CPAP hose holder is described herein as a separate system, it is meant to be able to function as a part of a system that is comprised of:
a) An example embodiment of the present disclosure.
b) A pillow.
c) A CPAP machine.
d) A CPAP hose.
e) A CPAP face mask.

The CPAP machine is connected to the CPAP face mask by means of a CPAP hose. This hose is placed inside an example embodiment of the present disclosure. The pillow is then placed on top of the CPAP hose holder.

An example embodiment of the present disclosure is made to fit under a pillow. The size of the base 2 and the total height of the support can be changed as needed to fit any pillow. There is no international standard pillow size. Many manufactures simply do not adhere to any dimensions. Table 1 gives the most common standard sizes for Europe and North America.

TABLE 1
standard pillow sizes for Europe and North America

| | Depth | Width |
|---|---|---|
| | (cm) | (cm) |
| Toddler & Travel | 41 | 51 |
| Regular | 51 | 66 |
| Standard | 51 | 71 |
| Queen | 51 | 76 |
| King | 51 | 91 |
| European | 66 | 66 |

These large variations in pillow sizes account for the variations in the dimensions of the example embodiments.

An example embodiment of the present disclosure is designed to fit a hose of a known diameter. Presently the standard for CPAP hoses is a 22 mm outer diameter. Thus supports for the present standard CPAP hoses should have an inner diameter of 22 mm. If this changes, the diameter of the supports of an example embodiment of the present disclosure could be changed to fit this new size.

The width of the base 2 can be 15 to 40 cm, more specifically 25 to 35 cm, and in one example 30 cm. The depth of the base 2 can be 10 to 30 cm, more specifically 15 to 25 cm, and in one example 20 cm.

The height of the lower support 3 together with the hose entryway 32 and lower connection means 31 can be 10 to 35 cm, more specifically 15 to 30 cm, and in one example 22.6 cm. The distance between the two hose entryways 32 of the lower support 3 can be 4 to 35 cm, more specifically 6 to 20 cm, and in one example 7.6 cm.

The height of the upper support 4 together with the platform 42 and the upper connection means 41 can be 10 to 30 cm, more specifically 15 to 25 cm, and in one example 18 cm. The width of the upper support can be 2 to 6 cm, more specifically 2.5 to 4 cm, and in one example 3 cm.

The height of the platform 42 of the upper support 4 can be 3 to 10 cm, more specifically 4 to 8 cm, and in one example 6.5 cm. The width of the platform 42 of the upper support 4 can be 2 to 15 cm, more specifically 4 to 10 cm, and in one example 5 cm. The platform 42 overhangs the top support 4 by between 1 and 15 cm, in one example 9.2 cm.

The total height of an example embodiment of the present invention, from bottom of base 2 to the top of the platform 42, can be 20 to 60 cm, more specifically 30 to 50 cm, and in one example 42 cm. The interior diameter of the supports of the present invention can be 15 to 30 mm, more specifically 20 to 26 mm, and in one example 22 mm

Optional embodiments may also be said to broadly include the parts, elements, steps and/or features referred to or indicated herein, individually or in any combination of two or more of the parts, elements, steps and/or features, and wherein specific integers are mentioned which have known equivalents in the art to which the disclosure relates, such known equivalents are deemed to be incorporated herein as if individually set forth.

The reference in this specification to any prior publication (or information derived from the prior publication), or to any matter which is known, is not, and should not be taken as an acknowledgment or admission or any form of suggestion that the prior publication (or information derived from the prior publication) or known matter forms part of the common general knowledge in the field of endeavor to which this specification relates.

Throughout this specification and the claims which follow, unless the context requires otherwise, the word “comprise”, and variations such as “comprised”, “comprises” or “comprising”, will be understood to imply the inclusion of a stated integer or step or group of integers or steps but not the exclusion of any other integer or step or group of integers or steps.

### LIST OF USED REFERENCE NUMERALS

1 CPAP Hose
2 Base
21 Base Hole
22 Base Connection Section
3 Lower Support
31 Lower Connection Means
32 Hose Entryway
33 Base Connection Means
34 Lower Hose Guide
35 Entryway Hose Guide
36 Lower Support Hole
4 Upper Support
41 Upper Connection Means
42 Platform
43 Upper Hose Guides
44 Upper Support Hole
45 Platform Hose Guide
46 Platform Hole

What is claimed is:

1. A CPAP hose holder device for the support of a CPAP hose comprising:
a base, a hose entryway with one or more openings for a CPAP hose, entryway hose guides, an elongated support, support hose guides, and a platform, wherein:
a bottom of the hose entryway is connected to the base, a top of the hose entryway is connected to a bottom of the support, and the platform is connected to a top of the support;
the entryway hose guides are connected to a back of the hose entryway, and the support hose guides are attached to a back of the support;
the hose guides and the support are arranged to partially or fully enclose the CPAP hose; and
the support is substantially upright with respect to the base, and the base and the platform extend from the support in substantially the same direction.

2. The CPAP hose holder device according to claim 1, wherein the support is comprised of two or more separate parts.

3. The CPAP hose holder device according to claim 1, wherein the support is comprised of two separate parts.

4. The CPAP hose holder device according to claim 1, wherein the support is straight.

5. The CPAP hose holder device according to claim 1, wherein the support is curved.

6. The CPAP hose holder device according to claim 1, wherein the CPAP hose holder device has a cover.

7. The CPAP hose holder device according to claim 1, wherein the CPAP hose holder device is arranged to be partially or wholly dissembled.

8. The CPAP hose holder device according to claim 1, wherein a pillow can be temporarily attached to the CPAP hose holder device.

9. Use of the CPAP hose holder device according to claim 1 for the prevention of face mask removal during sleep, wherein a hose from a CPAP machine is placed inside the CPAP hose holder device and a pillow is placed on the base of the CPAP hose holder device.

10. A method of moving the CPAP hose holder device according to claim 1 to another position in a bed while an individual is sleeping, comprising the following steps:
lifting the individual's head from a pillow;
moving the CPAP hose holder device and the pillow together to a new position in the bed;
placing the individual's head back upon the pillow.

11. A CPAP hose holder system comprised of:
the CPAP hose holder device according to claim 1;
a pillow;
a CPAP machine;
a CPAP hose; and
a CPAP facemask.
